(19) United States
(12) Patent Application Publication
(10) Pub. No.: US 2022/0228046 A1
(43) Pub. Date: Jul. 21, 2022

(54) COMPOSITION FOR DUST SUPPRESSION

(71) Applicant: W&C HOLDING B.V., Deinum (NL)

(72) Inventor: Wybren Jan BEEKSMA, Deinum (NL)

(21) Appl. No.: 17/611,337

(22) PCT Filed: May 7, 2020
(86) PCT No.: PCT/NL2020/050291

§ 371 Date: Nov. 15, 2021

(30) Foreign Application Priority Data
May 16, 2019 (NL) 2023148

(51) Int. Cl. C09K 3/22; C12N 1/20; C12N 11/10; C12N 1/34

(52) U.S. Cl. CPC C09K 3/22; C12N 1/20; C12R 2001/07; C12N 1/34; C12N 11/10

(57) ABSTRACT

The invention relates to an aqueous composition for the suppression of dust, comprising 1) an anionic surfactant; 2) an amphoteric surfactant; 3) a Bacillus species; and 4) a carbohydrate-based feedstock on which the Bacillus can grow. The composition is capable of forming a foam. When mixed with dust, the composition, in particular the foam thereof, is effective in preventing the dust from becoming airborne. Further, when applied before grinding or milling a material, the spreading of dust during and after the grinding or milling is effectively prevented.

The invention relates to a composition for dust suppression, to a foam comprising such composition, to a method for preparing such foam, to a dust comprising such foam or composition, to a method for suppressing dust and to a method for grinding or milling matter.

Processes wherein solid material is shaped, broken or separated are often accompanied with the generation of airborne dust, for example construction work, demolition work, mining, abrasive blasting (removal of paint or rust, cleaning of surfaces) and the like. Dust often also becomes airborne in the treatment of (demolition) waste, in industrial production processes (processing of food) and in agriculture (treatment of soil, intensive animal husbandry).

Such processes pose environmental issues in populated or environmentally sensitive areas. In particular, airborne dusts are of concern because they are associated with classical widespread occupational lung diseases (in particular pneumoconiosis) and diseases such as cancer, asthma, allergic alveolitis and irritation.

Many solutions to combat airborne dust aim at preventing the escape of dust when it is formed as a by-product. For example, the surface of coal may be treated with a particular dust-suppressing composition during the mining or the transport of the coal. Other solutions are focused on scavenging dust once released, for example from uncontrolled and/or widespread sources, such as traffic. A solution may then be to provide a coating on surfaces such as roads, pavements, and walls. These and other conventional solutions for dust-suppression typically rely on increasing the tackiness of the dust and the surfaces, respectively.

In contrast to the mere presence of dust as a by-product, there are also situations wherein the dust as such is a product of an industrial process and is collected in large quantities, for example when stony material is crushed into smaller pieces during e.g. demolition work or certain mining activities. A large fraction of such crushed material typically has a diameter of less than 1 mm and comprises a lot of dust. In such cases, it is more effective (and often even a necessity) to treat the dust product itself rather than neighboring surfaces on which the dust may land.

There are at present however no satisfactory treatments of dust as such that reduce the spreading of the dust into the environment. Methods that rely on tackiness are disadvantageous for this purpose, because this results in uncontrolled agglomeration of dust particles, which complicates the handling of the dust, for example on a conveyor belt or in a silo. Also, increased tackiness and agglomeration make sieving of the dust more difficult. Methods that rely on wetting the dust require large amounts of water and have the effect that the suppression of dust is only temporary (due to the evaporation of water).

It is therefore an objective of the present invention to provide a method for treating dust which on the one hand reduces its spreading into the environment and on the other hand does not lead to the difficulties in the handling of the dust as is the case with conventional methods, in particular which does not result in an uncontrolled agglomeration of dust particles.

It has now been found that one or more of these objectives can be reached by applying particular composition in the treatment of dust.

Accordingly, the present invention relates to a composition for the suppression of dust, comprising
an anionic surfactant;
an amphoteric surfactant;
one or more Bacillus species;
a carbohydrate-based feedstock on which the Bacillus can grow;
   wherein the composition is capable of forming a foam.

Dust is usually regarded as a collection of solid particles ranging in size from 0.2-200 μm, which may be or become airborne, depending on their origin, physical characteristics and ambient conditions. Dust may be present between larger chunks of material, i.e. between particles that have a diameter of at least 200 μm, for example with a diameter in the range of 0.5-100 mm. Typically, the smallest particle sizes in a particle size distribution of dusty solid matter correspond to those of dust. In principle, dust may be present in solid matter with any particle size distribution. The fraction of dust in a batch of solid matter may be in the range of 0.1-99.9 wt. %. For example, the dust fraction is in the range of 0.5-50 wt. % or in the range of 1.0-10 wt. %, as may be the case with ground stony matter such as ore or demolition waste.

There are several materials that may be present as dust. Such materials are usually solid. For example, the material is selected from the group of stone, sand, ore, glass, (dried) plant material, plastic, metal, yarns, textiles, cellulose and food products (e.g. milk powder, sugar, flour, powdered cereals). In general, the invention may be applied on any material that may drift and easily becomes airborn and/or on any material that may behave as a drifting dust.

Dust typically has the property that it is easily spread and/or displaced by moving air such as wind. When this occurs, the dust may become airborne. In particular, wind may be generated during handling of the dust, for example by transferring it from one container into another.

By the dry matter content of a composition of the invention is meant all constituents of the composition except the water and an eventual other solvent that is present.

An aqueous composition of the invention comprises water as a solvent. Usually, water is the only solvent present. However, it is in principle also possible that an additional solvent is present in the composition. Such solvent is preferably miscible with water, such as methanol, ethanol or acetone.

An aqueous composition of the invention is capable of forming a foam. This means that pockets of gas are trapped in the aqueous composition, yielding a substance of a significantly lower density than the original aqueous composition. Therefore, the aqueous composition comprises one or more foam-former. Typically, the foam-forming capacity of the composition originates from the surfactants that are present. Preferably, the anionic surfactant is a foam-forming surfactant; when two or more anionic surfactants are present in the composition, then at least one of them is usually a foam-forming surfactant. The amphoteric surfactant may also be a foam-forming surfactant; when two or more amphoteric surfactants are present in the composition, then at least one of them is usually a foam-forming surfactant. In any case, however, a person skilled in the art knows how to arrive at an aqueous composition that is capable of forming a foam. Given any combination of desired components in the aqueous composition of the invention, he will be able to make a composition of the invention that is capable of forming a foam by routine experimentation without exerting inventive effort.

The anionic surfactant may be a surfactant wherein a sulfonate group is connected to a hydrophobic chain. In particular, the anionic surfactant is an olefin sulfonate, more in particular an alpha-olefin sulfonate. For example, the alpha-olefin sulfonate has a C10, C12, C14, C16, C18 or C20 olefinic chain. The anionic surfactant may also comprise an alkylbenzene sulfonate. The benzene group may be substituted with at least one linear or branched alkyl group. For example, it is a linear alkylbenzene sulfonate comprising e.g. one or two C10, C12, C14, C16, C18 or C20 alkyl chains. Such anionic surfactant may be sodium dodecylbenzenesulfonate. The anionic surfactant may also be a branched alkylbenzene sulfonate comprising e.g. one or two chains of propylene oligomers (tetramers or pentamers).

There may also be more than one anionic surfactant present in a composition of the invention (i.e. at least two anionic surfactants), for example two, three or four anionic surfactants. In total, the anionic surfactant(s) usually constitute 35-65 wt. % of the total composition, based on the dry matter content, in particular 45-60 wt. %.

A composition of the invention also comprises an amphoteric surfactant. Typically, the amphoteric surfactant is an alkyliminodipropionate, such as sodium lauriminodipropionate. The amphoteric surfactant may also be betaine (trimethylglycine) or a substituted betaine. For example, the substituted betaine is an alkyl dimethyl betaine, in particular a C12 or C14 dimethyl betaine.

There may also be more than one amphoteric surfactant present in the composition of the invention, for example two, three or four amphoteric surfactants. In total, the amphoteric surfactant(s) usually constitute 4.0-40 wt. % of the total composition, based on the dry matter content, in particular 15-30 wt. %.

A composition of the invention optionally comprises one or more nonionic surfactants, for example a fatty acid ethoxylate or an alcohol ethoxylate. In particular, a nonionic surfactant is a sugar-based surfactant, which is a surfactant wherein the hydrophilic moiety is formed by a sugar moiety. To such sugar moiety is then attached a hydrophobic moiety, such as an alkyl chain. In total, the optional nonionic surfactant(s) usually constitute 10-35 wt. % of the total composition, based on the dry matter content, in particular 14-28 wt. %.

A composition of the invention optionally comprises urea or an urea derivative such as an N-alkyl urea or an N-aryl urea.

A composition of the invention also comprises a species of Bacillus. Typically, it comprises a blend of Bacillus species. For example, it comprises one or more strains or spores of one or more Bacillus species selected from the group of Bacillus subtilis, Bacillus amyloliquefaciens, Bacillus megaterium, Bacillus licheniformis, Bacillus atropheaus, Bacillus simplex, Bacillus velezensis, Bacillus cereus and Bacillus pumilus.

A composition of the invention also comprises a carbohydrate-based feedstock on which the one or more Bacillus species can grow. This feedstock is for example a sugar selected from the group of monosaccharides (such as glucose, galactose, fructose, xylose), disaccharides (such as sucrose, lactose, maltose, trehalose) and polyols (such as sorbitol, mannitol). It may also be a (malto-)oligosaccharide (such as maltodextrin, raffinose) or a sugar that is bound to another functional group via a glycosidic bond, such as a glucoside (e.g. alkyl glucosides such as decyl glucoside or lauryl glucoside) or a fructoside.

In total, the carbohydrate-based feedstock, in particular a glucoside, usually constitutes 5-40 wt. % of the total composition, based on the dry matter content, in particular 14-28 wt. %.

The one or more Bacillus species in a composition of the invention may be Bacillus spores. Such spores can remain in their dormant and resistant states for long periods of time. Exposure to essential agents can return the spores to life within minutes (germination). Such germination process converts the germinated spore into a growing cell.

When present in a composition of the invention, spores of Bacillus typically remain inactive (i.e. in a dormant state) until they are accessed by (atmospheric) oxygen. This usually occurs upon application of the composition on dust or matter to be milled/ground and/or when a foam of the composition is prepared. The spores will then germinate, consume the carbohydrate-based feedstock, grow and multiply.

It is contemplated that the effect of the carbohydrate-based feedstock is not only that the Bacillus can grow on it, but also that it forms a layer around the dust particles. The other components of the composition likely also end up in such layer. This includes the water, which would then add to the weight of the dust particles. It is contemplated that the Bacillus in such layer consumes the feedstock and so changes the composition of the layer. The result would be a layer that is not sticky and that has modified the properties of the dust particles in such manner that the dust is much less susceptible to becoming airborne. It is contemplated that this is due to e.g. an increased weight or modified aerodynamic properties of the dust particles.

The amount of carbohydrate-based feedstock in a composition of the invention is preferably 4.0 wt. % or less, more preferably 2.5 wt. % or less, e.g. in the range of 0.5-2.0 wt. % (i.e. based on the total composition, including the water). This is because the handling of the composition at higher concentrations becomes more difficult (stickiness, pumpability) and the treated dust becomes too sticky. When the composition is actually applied to suppress dust, the concentration of the carbohydrate-based feedstock is usually lower than 0.10 wt. %, preferably lower than 0.040 wt. %, more preferably lower than 0.025 wt. %. It may for example be lower than 0.015 wt. % or lower than 0.005 wt. %.

A composition of the invention may further comprise one or more additives, such as a preservative or a fragrance.

A composition of the invention usually has a pH of at least 5.0 or at least 6.0. Preferably, the pH is in the range of 6.0-11.0, more preferably in the range of 6.5-9.5. The pH may also be in the range of 9.5-11. A higher pH ensures that the equilibrium of the anionic surfactant with its conjugated acid is shifted enough towards deprotonation of the acid. On the other hand, a pH closer to neutral provides a better environment for the Bacillus to grow, for example a pH in the range of 7.0-9.0. Further, the pH of a composition of the invention is preferably at least one pH unit away from the isoelectric point of the amphoteric surfactant.

In a composition of the invention, the dry matter content is usually 80 wt. % or less. This means that water constitutes at least 20 wt. % of a composition of the invention. The dry matter content may also be 50 wt. % or less, 10 wt. % or less, 5 wt. % or less, 2 wt. % or less, 1 wt. % or less, 0.5 wt. % or less, 0.1 wt. % or less, 0.05 wt. % or less or 0.02 wt. % or less. Water may constitute at least 50 wt. % of a composition of the invention, at least 90 wt. %, at least 95 wt. %, at least 98 wt. %, at least 99 wt. %, at least 99.9 wt. %, at least 99.95 wt. % or at least 99.98 wt. % of the composition.

The content of the anionic surfactant is usually in the range of 35-65 wt. %, based on the dry matter content. The content of the amphoteric surfactant, in particular of the betaine or a derivative thereof, is usually in the range of 4-40 wt. %, based on the dry matter content. The content of the optional nonionic surfactant may be in the range of 14-28 wt. %, based on the dry matter content. The content of the carbohydrate-based feedstock is usually in the range of 5-40 wt. %, based on the dry matter content.

The composition is typically prepared, distributed and sold as a concentrate, having a dry matter content in the range of e.g. 2-20 wt % or 5-50 wt. %. Prior to its application in a method for the suppression of dust, such concentrate is diluted 50-200 fold, in particular 80-120 fold. For example, after dilution, the amount of water (including any other volatile, if present) in the composition is in the range of 99.00-99.99 wt. % (corresponding to a dry matter content in the range of 0.01-1.00 wt. %), or in the range of 99.50-99.98 wt. % (corresponding to a dry matter content in the range of 0.02-0.50 wt. %). Thus, a composition of the invention is relatively dilute, which allows an efficient use of its components.

The invention further relates to a method for preparing a composition as described above, comprising mixing water with 1) the anionic surfactant; 2) the amphoteric surfactant; 3) the optional nonionic surfactant; 4) one or more Bacillus species; and 5) the carbohydrate-based feedstock on which the Bacillus species can grow. The pH is optionally adjusted to a value in the range of 7.0-11.0. This may be performed by providing the water with a pH buffer, or by application of an appropriate amount of acid or base during or after the mixing.

A composition of the invention may be present as a foam. This means that pockets of gas, usually of air, are trapped in the aqueous composition so that its density is lower than that of the composition.

Usually, when a foam is prepared from the composition, the composition is relatively dilute (e.g. as compared to compositions applied in a non-foamed (i.e. liquid) state). For example, the water content in a foam is at least 95 wt. %, at least 99 wt. %, at least 99.5 wt. %, at least 99.9 wt. %, at least 99.95 wt. %, at least 99.98 wt. %, at least 99.99 wt. %, at least 99.995 wt. %, at least 99.998 wt. % or at least 99.999 wt. %. The dry matter content may be less than 10 wt. %, less than 5 wt. %, less than 1 wt. %, less than 0.5 wt. %, less than 0.1 wt. %, less than 0.05 wt. %, less than 0.02 wt. %, less than 0.01 wt. %, less than 0.005 wt. %, less than 0.002 wt. % or less than 0.001 wt. %.

The density of a foam of the invention is usually in the range of 1-200 kg/m³, in particular in the range of 5-100 kg/m³, more in particular in the range of 10-50 kg/m³.

The presence of the composition as a foam allows that the composition can be conveniently and evenly dispersed into a large amount of dust, preferably actively mixed therewith. Moreover, since dry matter contents as low as 0.10-0.001 wt. % can be used, the amount of surfactants, Bacillus and carbohydrate-based feedstock that is actually applied in the treatment of a particular amount of dust, is rather low. Thus, a composition of the invention allows a particularly efficient use of its components when it is in the form of a foam.

The invention further relates to a method for preparing a foam as described above, comprising providing a composition as described above and mixing the composition with air (aerating).

A foam of the invention may be prepared by feeding the aqueous composition of the invention together with air into a vessel (e.g. an expansion vessel or a pressure vessel) and shaking the vessel. The dry matter content of the composition that is used in the method is preferably in the range of 0.002-0.75 wt. %, 0.02-0.75 wt. % or in the range of 0.05-0.50 wt. %.

In the method for preparing the foam, the composition typically
has a dry matter content in the range of 0.10-1.00 wt. %, in particular in the range of 0.20-0.50 wt. %; and
is further diluted by adding at least 50 parts of water prior to mixing it with air, preferably 90-120 parts of water.

The invention further relates to dust comprising a composition as described above, including dust comprising a foam that is prepared from such composition.

Generally, in case dust is present as a bulk substance, the skilled person would consider that the use of a dust-suppressant concerns the treatment of a certain amount of dust, while in case of a very thin layer of dust residing on a surface, he would consider that it concerns the treatment of the surface (such as a road surface). Prior art on the suppression of dust usually concerns the treatment of a surface on which dust is present or will be present later on. This is for example the case in WO2014198840A1, where a dust suppressant composition is disclosed which is used on surfaces such as roads.

In the case of the invention, the composition or foam has been found to be most effective in dust suppression, when the dust is concentrated. This means that the dust has the appearance of a powdery substance in bulk rather than that it is spread as a very thin layer over a large surface such as a road.

Thus, dust of the invention is preferably present in bulk quantities. Accordingly, the invention further relates to a container comprising the dust as described above, typically a container that is filled with dust to a height of at least 0.5 cm, at least 1.0 cm, at least 2.0 cm, at least 3.0 cm or at least 5.0 cm. In particular, on at least one position on a bottom of the container, that position is covered with at least 0.5 cm, at least 1.0 cm, at least 2.0 cm, at least 3.0 cm or at least 5.0 cm of the dust. By a container is e.g. meant an element selected from the group of boxes, bins, trays, buckets and jars. Also a scoop and a conveyor belt are included, since these can also hold the dust. Depending on the application, the container may be capable of being closed off from the environment by e.g. a lid or a cover.

The effect of the composition on the dust is likely that the dust becomes heavier, or at least that it quickly deposits rather than that it stays air-borne. This is not the result of the agglomeration of dust particles, neither have dust particles become tacky due to the composition. This is supported by the surprising observation that the dust may still be sieved, and does not block the sieve (thus, typically, prior to the sieving, all of the dust that has to be sieved has been treated with a composition of the invention in advance). This advantageous effect accounts for all kinds of sieves, e.g. sieves that can separate any two or more particle sizes, for example sieves that only allow particles with sizes of less than 2 mm or less than 0.5 mm to pass.

A dust of the invention makes sieving the dust much more convenient than the sieving of other dusts, since less efforts (or none at all) have to be made to prevent the dust from becoming airborne during the sieving. Likely, these advantageous effects are due to the presence of Bacillus in the composition.

A foam or composition of the invention is advantageously applied on dust, so that dust is prevented from spreading in the environment, in particular from becoming airborne. Accordingly, the present invention further relates to a method for suppressing dust, comprising
providing dust; then
contacting the dust with a composition as described above, in particular with a foam that is prepared from such composition; then
optionally mixing the dust with the composition, in particular with the foam that is prepared from such composition.

Thus, in the method, the dust is contacted with the composition, e.g. by spraying the composition onto the dust. Preferably, however, the composition is also mixed with the dust, more preferably it is thoroughly mixed. By mixing is meant that both components are combined and blended into one mass, for example by scooping the dust, by (repeatedly) pouring it from one container into another or by using a blender or a mixer.

In a method of the invention, the dust is usually present in a container, preferably a container that is closed off from the environment so that any airborne dust generated during the method cannot escape to the environment.

The dust that is contacted with the composition typically comprises particles with a size of 0.20 mm or less. The dust may also comprise particles with a size of more than 0.20 mm, in particular of more than 1.0 mm or more than 10 mm. For example, the fraction of particles with a size of more than 0.20 mm is in the range of 0.1-50 wt. % or in the range of 1.0-10 wt. %. The fraction of particles with a size of 0.20 mm or less may constitute 0.1-99.9 wt. %, in particular 1-99 wt. %, more in particular 25-75 wt. %. Particles with a size of less than 0.20 mm may also constitute 50-99.9 wt. %.

As already explained above, the suppression of dust according to the invention is based on the treatment of dust (in bulk) with an aqueous composition of the invention. This is in contrast to the treatments often described in the art, wherein a particular surface is treated with the aim to suppress dust accidentally passing the treated surface.

Further, when applied in this way, the use of the composition is much more efficient than when a particular surface is treated, since the mass ratio of composition:dust used in the method can be kept low whilst still providing sufficient dust suppression.

Typically, the mass ratio of the “applied composition on a dry matter basis”:“dust to be treated” is in the range of 1:10,000 to 1:50,000,000, in particular in the range of 1:100,000 to 1:10,000,000, more in particular in the range of 1:500,000 to 1:5,000,000. Given the typical dry matter contents of the compositions, the mass ratio of the “applied composition as such (i.e. including the water)”:“dust to be treated” is typically in the range of 1:10 to 1:50,000, in particular in the range of 1:50 to 1:10,000, more in particular in the range of 1:100 to 1:5,000. The “dust to be treated” as referred to above may also include particles with a size of more than 0.20 mm (besides the dust particles with a size of 0.20 mm or less), for example in an amount of 0.1-50 wt. %, in an amount of 10-40 wt. % or in an amount of 20-60 wt. %.

It was found that the use of the composition as a foam has advantages over the use of a non-foamed composition. First, when mixed with dust, it is easier to reach a homogeneous distribution of the composition over the dust when the composition is applied in the form of a foam. Second, the dosing of the composition to the dust is easier when it is in the form of a foam, since the mass ratio of composition : dust is rather low (in contrast to the volume ratio).

The method of the invention is particularly useful when dust requires sieving (e.g. separating dust particles based on their size). Sieving of dust often results in lots of dust becoming airborne, which is highly undesired. The method of the invention is particularly useful and advantageous to suppress such dust release. The advantages manifest when the sieving is preceded by treating the dust according to the method of the invention. Accordingly, the method of the invention is preferably followed by sieving the dust.

The composition (or foam) of the invention may also be applied before dust is actually generated, for example prior to processes that reduce the particle size of a material such as milling or grinding the material. This has the advantage that, during the reduction (in particular during the milling or grinding), dust does not get a chance of becoming airborne. Accordingly, the invention further relates to a method for reducing the particle size of matter, in particular for grinding or milling matter, wherein dust formation is suppressed, comprising
applying a foam or composition as described above to the matter that needs particle size reduction, in particular to the matter that needs to be ground or milled; then
reducing the particle size of the resulting matter, in particular grinding or milling the resulting matter.

When the foam or composition is used in a method for dust suppression or in a method for reducing the particle size of matter (by e.g. grinding/milling the matter) as described above, then the foam or composition is provided on the dust or the matter, respectively. It then serves as a carrier to provide a good distribution of the components of the composition. This adds to efficient use of the composition since more dust can be effectively treated with the same amount of composition. In principle, application of the composition alone (i.e. non-foamed) provides the advantages of dust suppression, but usually better effects are obtained when the foam is applied.

Typically, the mass ratio of the “applied composition on a dry matter basis”:“matter that is to be ground or milled” is in the range of 1:10,000 to 1:50,000,000, in particular in the range of 1:100,000 to 1:10,000,000, more in particular in the range of 1:500,000 to 1:5,000,000. Given the typical dry matter contents of the compositions, the mass ratio of the “applied composition as such (i.e. including the water)”:“matter that needs to be ground or milled” is typically in the range of 1:10 to 1:50,000, in particular in the range of 1:50 to 1:10,000, more in particular in the range of 1:100 to 1:5,000.

After completion of the dust suppression methods with the foam as described above (including the grinding/milling method), the foam is usually not present as a foam anymore. Instead, the composition is dispersed over the dust particles and has changed into a non-sticky layer around the dust particles, aided by the action of the Bacillus. It is contemplated that the layer also contains some of the water that is present in the composition, and that such layer largely prevents its evaporation, yielding dust with heavier particles.

In the method for suppressing dust or in the method for grinding or milling matter, the amount of carbohydrate-based feedstock in the foam or composition is usually in the range of 0.0025-0.025 wt. % of the composition or the foam. With regard to the foam, the wt. % is then based on the solid and liquid components of the foam (gas contained in the foam is excluded).

A further advantage of a composition or a foam of the invention is that all components may be chosen such that the composition or foam is biologically degradable and at the same time highly effective. This improves the conditions of workers and operators in places where the invention is applied, as well as reduces the impact on the environment.

### EXAMPLES

### Experimental Set-Up

The amount of dust formation with and without the dust-suppressing composition of the invention was determined with the following set-up. Pieces of stony material are collected on a conveyor belt. Halfway the conveyor belt, eight nozzles (capacity number BF 6530) with an equivalent bore with a diameter of 3.28 mm were positioned above the belt, designed to dose the foam composition of the invention under an angle of 50° with the belt in the direction of translation of the belt. The end part of the conveyor belt is arranged above a dumpster, allowing the material to drop into the dumpster and to collect in the dumpster. The throughput of the material is 60 tons of per hour. The dumpster was covered with plastic, leaving only a small opening for the material to enter the dumpster. In this way, a more or less closed compartment is formed above the surface of the material that is collected in the dumpster. In the compartment, a fine dust measuring device (Fidas® 200 S connected to a GilAir-3 Air Sampling Pump) was placed that can quantitatively measure the presence of PM 10 and PM 2.5 dust, which is defined as particulate matter with a particle size of 10 μm and 2.5 μm, respectively.

### Experimental Procedure

The starting material for the process is formed by a dry mixture of 400 kg of debris with 25 kg of cement. The aqueous composition of the invention comprises, based on the dry matter content,
40 wt. % of sodium C14-16 olefin sulfonate (CAS Number: 68439-57-6);
15 wt. % of alcohols, C12-14, ethoxylated, sulfates, sodium salts (CAS Number: 68891-38-3);
20 wt. % of betaine (CAS Number: 107-43-7);
1 wt. % of a blend of Bacillus spores;
24 wt. % of laurylglycoside (CAS Number: 110615-47-9).

Just prior to its conversion into a foam and its application as a dust-suppressant, a concentrate of the composition with a dry matter content of 0.5 wt. % is diluted 100-fold.

General procedure: the cement-debris mixture is poured onto the vibrating screen and the matter that passes the screen is collected at the moving conveyor belt. During the transport of the matter over the belt, foam of the invention may be dosed to the matter. When the matter arrives at the end of this belt, it falls into the opening above the dumpster, passes the compartment with the fine dust measuring device and collects in the dumpster. During this process, the concentration of dust particles is measured in the atmosphere of the compartment.

Two different runs were performed; a first run wherein no means for the suppression of dust were applied (comparative run) and a second run wherein a composition of the invention was applied (dust suppressing run). The latter was performed by spraying the composition of the invention as a foam on the matter on the moving conveyor belt. The consumption of the composition of the invention was 3 kg per hour and the consumption of water as the diluent was 300 L per hour. Thus, given that 0.015 kg of composition, based on the dry matter content, is used to treat 60 tons of material, the mass ratio of the applied composition to the treated material is 1:4,000,000. The mass ratio of the applied composition as such (i.e. including the water) to the treated material is 1:198. The air pressure in the nozzles was 0.75 bars. The experiments were carried out in open air under dry, sunny, and windless weather conditions.

### Results

During both runs, the concentration of dust (PM 10 and PM 2.5) was measured in the air of the compartment. The results are displayed in Table 1; the displayed concentrations are the average of two measurements.

TABLE 1
Measured concentrations of PM 10 and PM 2.5

| | Concentration of | Concentration of |
|---|---|---|
| | PM 10 (mg m⁻³) | PM 2.5 (mg m⁻³) |
| First run (reference run) | 19.3 | 0.83 |
| Second run (invention run) | 1.7 | 0.47 |

### CONCLUSIONS

The concentration of PM 10 is decreased by a factor of 11.4 and the concentration of PM 2.5 by a factor of 1.8. Thus, a dust suppression method of the invention is highly effective.

1. Aqueous composition for the suppression of dust, comprising
an anionic surfactant;
an amphoteric surfactant;
a Bacillus species;
a carbohydrate-based feedstock on which the Bacillus can grow;
wherein the composition is capable of forming a foam.

2. Composition according to claim 1, wherein the anionic surfactant comprises one or more surfactants selected from the group of C10-C20 alpha-olefin sulfonates, linear alkylbenzene sulfonates and branched alkylbenzene sulfonates.

3. Composition according to claim 1, wherein the anionic surfactant constitutes 35-65 wt. % of the total composition, based on the dry matter content, in particular 45-60 wt. %.

4. Composition according to claim 1, wherein the amphoteric surfactant comprises an amphoteric surfactant selected from the group of alkyliminodipropionates, betaine and substituted betaine such as a C12 or C14 dimethyl betaine.

5. Composition according to claim 1, comprising one or more strains or spores of a Bacillus species selected from the group of Bacillus subtilis, Bacillus amyloliquefaciens, Bacillus megaterium, Bacillus licheniformis, Bacillus atropheaus, Bacillus simplex, Bacillus velezensis, Bacillus cereus and Bacillus pumilus.

6. Composition according to claim 1, wherein the carbohydrate-based feedstock comprises a glucoside, in particular an alkyl glucoside such as decyl glucoside or lauryl glucoside.

7. Composition according to claim 1, wherein the dry matter content is in the range of 0.02-1.00 wt. %, in particular in the range of 0.02-0.50 wt. %.

8. A foam comprising the composition according to claim 1, wherein the foam has a density in the range of 1-200 kg/m³, in particular in the range of 5-100 kg/m³, more in particular in the range of 10-50 kg/m³.

9. Dust comprising a composition according to claim 1.

10. Dust comprising a foam of claim 8.

11. Method for preparing a foam according to claim 9, comprising
providing a composition; then
mixing the composition with air.

12. Method according to claim 11, wherein the composition
has a dry matter content in the range of 0.02-1.00 wt. %, in particular in the range of 0.02-0.50 wt. %; and
is further diluted by adding at least 50 parts of water prior to mixing it with air, preferably 9 120 parts of water.

13. Method for suppressing dust and for preparing a dust according to claim 9, comprising
providing dust; then
mixing the dust with a composition.

14. Method according to claim 13, wherein the composition is a foam according to claim 8.

15. Method according to claim 13, wherein the method is followed by sieving the dust.

16. Method for grinding or milling matter and preparing a dust according to claim 9, comprising
applying a foam to the matter that needs to be ground or milled; then
grinding or milling the resulting matter.

17. Method according to claim 13, wherein the amount of carbohydrate-based feedstock in the foam or composition is in the range of 0.0025-0.025 wt. % of the composition or the foam.
